(19) United States
(12) Patent Application Publication
(10) Pub. No.: US 2007/0231800 A1
(43) Pub. Date: Oct. 4, 2007

(54) Determination of methylated DNA

(75) Inventors: Douglas N. Roberts, Campbell, CA (US); Anniek De Witte, Palo Alto, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Loveland, CO (US)

(21) Appl. No.: 11/390,828

(22) Filed: Mar. 28, 2006

(51) Int. Cl. C12Q 1/68; C12P 19/34

(52) U.S. Cl. 435/6; 435/91.2

(57) ABSTRACT

The present invention generally relates to the determination of the state of one or more locations within a nucleic acid and, in particular, to the determination of the methylation state of one or more methylation sites within a nucleic acid such as DNA. In one aspect of the invention, a nucleic acid, such as DNA, that is suspected of being methylated is exposed to a nucleic acid probe able to hybridize the nucleic acid at or near the methylation site. After hybridization, the nucleic acid-probe hybrid is exposed to a methylation-sensitive restriction endonuclease able to bind at or near the methylation site. The restriction endonuclease is not able to cleave the nucleic acid-probe hybrid if the DNA is methylated at the methylation site, but is able to cleave the nucleic acid-probe hybrid if the nucleic acid is not methylated at the methylation site. Determination of the cleavage state of the probe can thus be used to determine the state of the methylation site.

### BACKGROUND

Methylation of nucleotides in DNA serves a number of cellular functions. In bacteria, methylation of cytosine and adenine residues plays a role in the regulation of DNA replication and repair. DNA methylation also constitutes part of an immune mechanism that allows these bacteria to distinguish between self and non-self DNA. In mammalian species, DNA methylation typically occurs at cytosine residues, and usually at cytosine residues that occur next to a guanosine residue, i.e., within the sequence CpG.

Methylation of DNA is typically performed by enzymes known as methyltransferases (also sometimes called methylases). Generally, both strands of a DNA duplex can accept methyl groups at opposing CpG sites, as CpG is self-complementary. Replication of a DNA duplex in which both strands have been methylated yields two new “hemi-methylated” DNA duplexes, each of which includes one of the methylated DNA strands of the original duplex and one newly-synthesized DNA strand that is not methylated. Certain maintenance enzymes, known as methyltransferases, are then able to restore full methylation to both strands of the newly-formed DNA duplexes.

Many CpG sites within a genome are found in a methylated state, and some CpG sites occur near coding regions within the genome. Such methylation has been linked to gene expression. Additionally, alterations in DNA methylation within a genome often are a manifestation of genomic instability, which may be a characteristic sign of a tumor. Thus, techniques for determining the methylation of DNA finds use in many different applications.

### SUMMARY OF THE INVENTION

The present invention generally relates to the determination of the state of one or more locations within a nucleic acid and, in particular, to the determination of the methylation state of one or more methylation sites within a nucleic acid such as DNA. The subject matter of the present invention involves, in some cases, interrelated products, alternative solutions to a particular problem, and/or a plurality of different uses of one or more systems and/or articles.

In one aspect, the invention is directed to a method of determining methylation of a nucleic acid molecule. The method includes, in one set of embodiments, acts of providing a nucleic acid molecule suspected of being methylated at a methylation site, hybridizing a nucleic acid probe to the nucleic acid molecule proximate the methylation site to produce a nucleic acid molecule-nucleic acid probe hybrid, exposing the nucleic acid molecule-nucleic acid probe hybrid to a methylation-sensitive restriction endonuclease, and determining a cleavage state of the nucleic acid probe to determine methylation of the nucleic acid at the methylation site.

In another set of embodiments, the method includes acts of exposing a nucleic acid molecule to a surface having at least a first region comprising a first nucleic acid probe immobilized thereto and a second region comprising a second nucleic acid probe immobilized thereto, where the first nucleic acid probe is able to hybridize the nucleic acid molecule at a first region suspected of being methylated at a first methylation site, and the second nucleic acid probe is able to hybridize the nucleic acid molecule at a second region suspected of being methylated at a second methylation site different from the first methylation site, exposing at least one of the first nucleic acid probe and the second nucleic acid probe to a restriction endonuclease, and determining a cleavage state of the first nucleic acid probe and/or the second nucleic acid probe to determine, respectively, methylation of the nucleic acid at the first methylation site and/or the second methylation site.

In yet another aspect, the invention contemplates a method of determining the state of a target site of nucleic acid. In one set of embodiments, the method includes acts of providing a nucleic acid molecule having a target site that can be in one of a plurality of naturally-occurring states, including a first state and a second state, hybridizing a nucleic acid probe to the nucleic acid molecule proximate the target site, exposing the nucleic acid-nucleic acid probe hybrid to a restriction endonuclease that does not bind the nucleic acid molecule if the target site is in a first state, but does bind the nucleic acid if the target site is in a second state, and thereafter, determining a cleavage state of the nucleic acid probe to determine the state of the target site.

In another aspect, the present invention is directed to a method of making or using one or more of the embodiments described herein, for example, a method of determining methylation of DNA. Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B schematically illustrate an assay to determine methylation of a nucleic acid, according to one embodiment of the invention;

FIGS. 2A-2B illustrate various probes useful in certain aspects of the invention;

FIG. 3 illustrates, as a non-limiting example, a portion of a genomic DNA sequence that can be studied according to one embodiment of the invention;

FIGS. 4A-4B illustrate the sequence shown in FIG. 3 having various nucleic acid probes of the invention hybridized to it;

FIGS. 5A-5B show the sequences of mouse DNMT1 and HpaII, respectively; and

FIGS. 6A-6B schematically illustrate an assay to determine methylation of a nucleic acid, according to another embodiment of the invention.

### BRIEF DESCRIPTION OF THE SEQUENCES

SEQ ID NO: 1 is ATCTCCCAGTGGCGCAGATACGCTCCGGCCCACCCGCCC, a synthetic sequence used within a nucleic acid probe in one embodiment of the invention;

SEQ ID NO: 2 is TCCGGCCCACCCGCCCGGCAGTCGAGGCGGACCCCTCCC, a synthetic sequence used within a nucleic acid probe in another embodiment of the invention;

SEQ ID NO: 3 is TAGAGGGTCACCGCGTCTATGCGAGGCCGGGTGGGCGGGCCGTCAGCTCCGC CTGGGGAGGGGTCCGCGC, a portion of a genomic DNA sequence that can be studied according to one embodiment of the invention;

SEQ ID NO: 4 is the amino acid sequence of mouse DNMT1, useful in certain embodiments of the invention; and

SEQ ID NO: 5 is the amino acid sequence of the methylation-sensitive restriction endonuclease HpaII, useful in certain embodiments of the invention.

### DETAILED DESCRIPTION

DNA is a molecule that is present within all living cells. DNA encodes genetic instructions which tell the cell what to do. By “examining” the instructions, the cell can produce certain proteins or molecules, or perform various activities. DNA itself is a long, linear molecule where the genetic information is encoded using any one of four possible “bases,” or molecular units, in each position along the DNA. This is roughly analogous to “beads on a string,” where a string may have a large number of beads on it, encoding various types of information, although each bead along the string can only be of one of four different colors.

In some cases, however, the cell may “methylate” a base on the DNA, which is a chemical reaction that subtly alters the base in a way that the cell can later recognize it. This may be performed for various reasons, such as to indicate that a particular piece of information is no longer important to the cell. The cell may also “demethylate” the base in some cases, e.g., to indicate that the information is again important to the cell. Extending the above “beads on a string” analogy, this would be akin to marking a bead with a piece of tape, which could later be removed, if necessary.

Scientists who study cells are interested in observing which bases along a given piece of DNA have been methylated. This has important implications in fields such as cancer research or research into hereditary diseases. However, as DNA is small and difficult to work with, scientists are interested in techniques for discovering which bases along the DNA have been methylated. This invention discloses several novel techniques, below. In one of these techniques, DNA is attached to a surface, and a complementary “probe” molecule that recognizes certain base sequences of the DNA is allowed to bind to the DNA to form a “complex” of the DNA and the probe. The complex is then exposed to another molecule (an enzyme) which is able to “cleave” or cut the complex into smaller fragments if the DNA at that location has not been methylated, but is not able to cut the complex if the DNA at that location has been methylated. By subsequently determining if the complex has been cut or is still intact, scientists can then determine whether the DNA at that location has been methylated.

More specifically, the present invention generally relates to the determination of the state of one or more locations within a nucleic acid and, in particular, to the determination of the methylation state of one or more methylation sites within a nucleic acid such as DNA. In one aspect of the invention, a nucleic acid, such as DNA, that is suspected of being methylated is exposed to a nucleic acid probe able to hybridize the nucleic acid at or near the methylation site. After hybridization, the nucleic acid-probe hybrid is exposed to a methylation-sensitive restriction endonuclease able to bind at or near the methylation site. The restriction endonuclease is not able to cleave the nucleic acid-probe hybrid if the DNA is methylated at the methylation site, but is able to cleave the nucleic acid-probe hybrid if the nucleic acid is not methylated at the methylation site. Determination of the cleavage state of the probe can thus be used to determine the state of the methylation site. In some cases, the probe may be immobilized with respect to a surface, such as the surface of an array. Other aspects of the invention are directed to methods of determining the state of one or more locations within a nucleic acid, for example, by hybridizing the nucleic acid to a probe and exposing the nucleic acid-probe hybrid to a restriction endonuclease that does not cleave the probe if a site within the nucleic acid is in a first state, but does cleave the probe if the site within the nucleic acid is in a second state. Yet other aspects of the invention are directed to devices or kits for determining nucleic acid methylation or other states of the nucleic acid, methods of promoting such determinations, and the like.

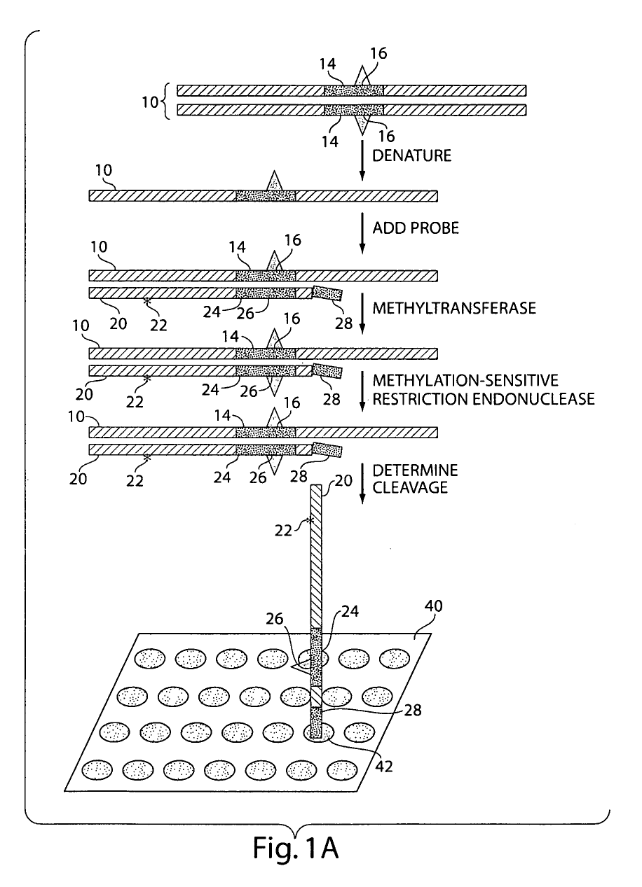

FIG. 1 illustrates an example of an assay according to one embodiment of the invention. A nucleic acid probe is used to determine whether a methylation site within a DNA strand has been methylated. Two cases are shown in FIG. 1. In FIG. 1A, the assay is performed on DNA in which a methylation site is methylated. In FIG. 1B, in contrast, the assay is performed on DNA in which the methylation site is not methylated. It should be noted that, in the following assay, an array is not necessarily required, and in other embodiments of the invention, the assay may be performed, for example, in solution.

As shown in FIGS. 1A and 1B, double-stranded DNA 10 is initially provided. However, this is by way of example only, and in other cases, single-stranded DNA, or other nucleic acids, may be provided instead. The nucleic acid may be any suitable nucleic acid which contains, or is suspected to contain, a methylation site. For example, the nucleic acid may arise from genomic DNA, mitochondrial DNA, cDNA, RNA, mRNA or the like, i.e., the source of the nucleic acid may be, for instance, genomic DNA, mitochondrial DNA, cDNA, RNA, mRNA, etc. In some embodiments, the nucleic acid may correspond to a chromosome, which may be non-cellular in some cases, as further described below. As shown in FIGS. 1A and 1B, DNA 10 includes a restriction site 14, and within restriction site 14, a methylation site 16, although methylation site 16 does not necessarily have to be contained within restriction site 14, as is discussed in more detail below. In FIG. 1A, methylation site 16 is shown as having been methylated (triangular markers), while in FIG. 1B, no triangular marker is present, indicating that methylation site 16 is not methylated. If the DNA (or other nucleic acid) is double-stranded, as is shown in FIGS. 1A and 1B, the DNA may be treated to render it single-stranded, for example, by denaturation or melting.

Next, DNA 10 is exposed to nucleic acid probe 20. Nucleic acid probe 20 includes detection entity 22, restriction site 24 including methylation site 26, and tag sequence 28. As above, methylation site 26 does not necessarily have to be contained within restriction site 24 of nucleic acid probe 20. At least a portion of nucleic acid probe 20 may be substantially complementary to DNA 10, and thus, these two strands can hybridize under suitable conditions, as is shown in FIGS. 1A and 1B. Thus, at least a portion of restriction sites 14 and 24 may be at least substantially complementary. In some cases, other portions of nucleic acid probe 20 may also be at least substantially complementary to DNA 10, for example, a portion of nucleic acid probe 20 in which detection entity 22 is located. Other portions of nucleic acid probe 20 do not have to be substantially complementary to DNA 10. As an example, as shown in FIGS. 1A and 1B, tag sequence 28 is not substantially complementary to DNA 10, and not able to hybridize with DNA 10.

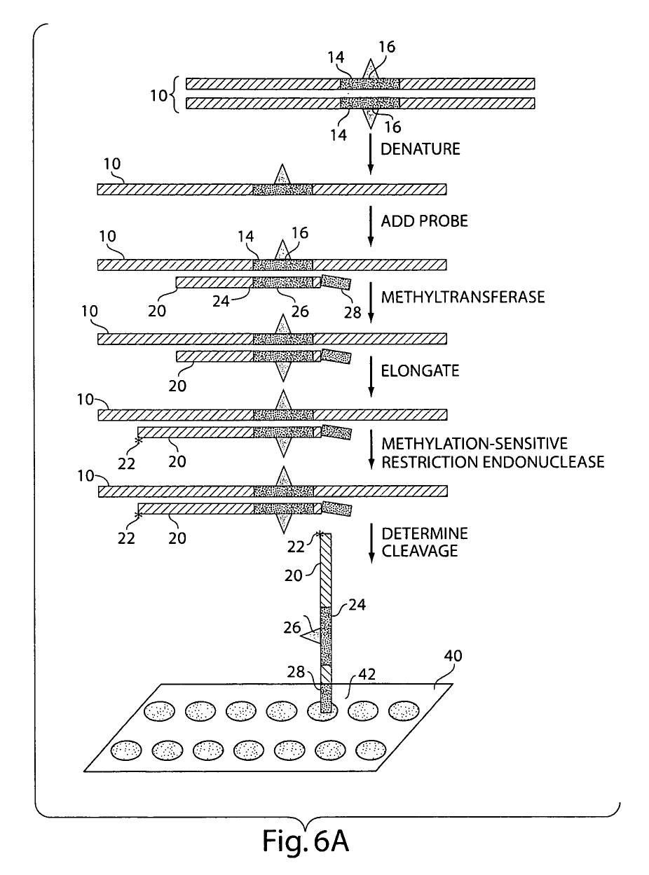

Optionally, the DNA-probe hybrid may be exposed to a methyltransferase, i.e., an enzyme able to catalyze the transfer (i.e., copying) of a methyl group located on one strand of a nucleic acid duplex or hybrid to the complimentary strand. Thus, a hemi-methylated DNA-probe hybrid, i.e., a hybrid in which one of the DNA strands is methylated, then becomes correspondingly methylated on the other strand, in approximately the same location. For example, a CpG site that is methylated on one strand will become correspondingly methylated on the other strand of the DNA duplex, as the CpG site is self-complimentary. A non-limiting example of a methyltransferase is DNMT1, for example, mouse DNMT1 (SEQ ID NO: 4, FIG. 5A). After exposure to the methyltransferase, as shown in FIG. 1A, methylation site 26 of nucleic acid probe 20 becomes methylated (indicated by the additional triangular marker on methylation site 26). In contrast, in FIG. 1B, since methylation site 16 of DNA 10 was not initially methylated, the methyltransferase is not able to alter nucleic acid probe 20, and thus, methylation site 26 of the nucleic acid probe remains unmethylated.

Next, the DNA-probe hybrid is exposed to a methylation-sensitive restriction endonuclease that is able to cleave the DNA-probe hybrid only if methylation sites 16 and/or 26 are not methylated. Examples of methylation-sensitive restriction endonucleases include, but are not limited to, HpaII (SEQ ID NO: 5, FIG. 5B) or AciI. In some cases, the methylation-sensitive restriction endonuclease is able to bind the DNA-probe hybrid if methylation sites 16 and/or 26 are not methylated, but is unable to cleave the DNA-probe hybrid. In other cases, the methylation-sensitive restriction endonuclease is not able to bind to the DNA-probe hybrid. Thus, in FIG. 1A, as both methylation sites 16 and 26 are methylated, the methylation-sensitive restriction endonuclease is not able to cleave the DNA-probe hybrid, and the hybrid thus remains unaltered. In contrast, in FIG. 1B, as both methylation sites 16 and 26 are not methylated, the methylation-sensitive restriction endonuclease is able to bind to and cleave the DNA-probe hybrid, as indicated by break 30. Each of nucleic acid probe 20 and DNA 10 is thus cleaved, forming separate fragments.

Afterwards, probe 20 is assessed to determine whether the probe was cleaved or not. One non-limiting method of assessing cleavage is illustrated in FIGS. 1A and 1B; other methods are described in more detail below. In this example, tag sequence 28 on nucleic acid probe 20 is substantially complementary to a nucleic acid immobilized with respect to the surface of array 40 at location 42. It should be noted that an array is not required to perform this assessment, and other techniques or surfaces that are not arrays may also be used in different embodiments. In this example, the DNA-probe hybrid may be denatured or melted to separate nucleic acid probe 20 from nucleic acid 10, and nucleic acid probe 20 is then exposed to the surface of array 40 (nucleic acid 10 may or may not be present during the exposure of nucleic acid probe 20 to the surface of array 40). Tag sequence 28 can become immobilized with respect to array 40 at location 42 by hybridizing to a substantially complementary nucleic acid immobilized at that location. Thus, in FIG. 1A, the entire nucleic acid probe 20, including detection entity 22, is localized to location 42; in FIG. 1B, in contrast, only a fragment of nucleic acid probe 20, i.e., the fragment of nucleic acid probe 20 containing tag sequence 28, can become immobilized with respect to location 42. In particular, it should be noted that this immobilizable fragment of nucleic acid probe 20 in FIG. 1B does not contain detection entity 22.

The presence or absence of detection entity 22 on array 40 with respect to location 42 can then be determined using any suitable technique. For example, if detection entity 22 is fluorescent, then a suitable method of detecting the fluorescence of location 42 may be used to determine the presence or absence of detection entity 22 with respect to that location. Non-limiting examples of such methods include a microarray plate reader, a spectrofluorimeter, etc. In some cases, other information may also be determined, for instance, the concentration and/or amount of nucleic acid probe 20 immobilized with respect to location 42, the immobilization of nucleic acid probe 20 with respect to other locations in array 40, etc., as further discussed in detail below.

Thus, in FIG. 1A, detection entity 22 is immobilized with respect to location 42, while in FIG. 1B, detection entity 22 is not immobilized with respect to location 42. By determining the presence and/or concentration of detection entity 22 with respect to location 42, information can be obtained as to whether methylation site 16 in DNA 10 was initially methylated or not. The immobilization of detection entity 22 with respect to location 42 indicates that DNA 10 was methylated at methylation site 16, while the absence (or a lower concentration or amount) of detection entity 22 with respect to location 42 indicates that DNA 10 was not methylated at methylation site 16. Of course, as mentioned, array 40 is not necessarily required, and in other embodiments of the invention, methylation may be determined, for example, by detecting fluorescence in solution.

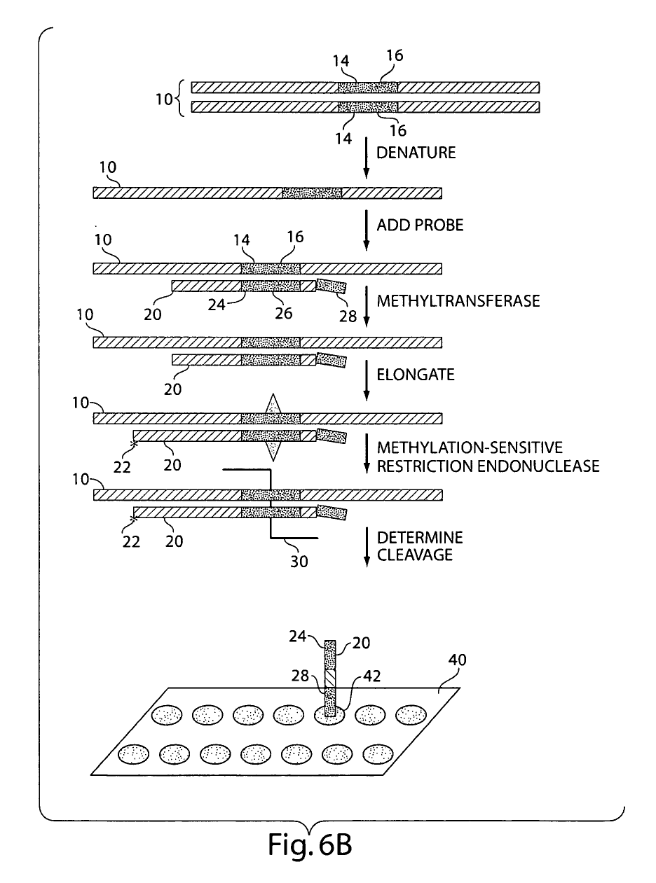

Another embodiment of the invention is illustrated in FIGS. 6A and 6B. As with FIGS. 1A and 1B, double-stranded DNA 10 is initially provided. DNA 10 includes a restriction site 14, and within restriction site 14, methylation site 16. In FIG. 6A, methylation site 16 is methylated (triangular markers), while in FIG. 6B, methylation site 16 is not methylated (no triangular marker). DNA 10 is then denatured to render it single-stranded.

Next, DNA 10 as exposed to nucleic acid probe 20, which includes restriction site 24 including methylation site 26, and tag sequence 28. At least a portion of nucleic acid probe 20 may be substantially complimentary to DNA 10, and thus, these strands may hybridize, as is shown in FIGS. 6A and 6B. Of course, as previously discussed, other portions of nucleic acid probe 20 do not have to be substantially complimentary to DNA 10, for example, tag sequence 28.

The DNA-probe hybrid may then be exposed to a methyltransferase, for example, DMMT1. After exposure to the methyltransferase, as is shown in FIG. 6A, methylation site 26 of nucleic acid probe 20 may become methylated (indicated by the additional triangular marker on nucleic acid probe 20). However, in FIG. 6B, since methylation site 16 of DNA 10 was not initially methylated, the methyltransferase is not able to alter nucleic acid probe 20.

Next, the DNA-probe hybrid may be exposed to an enzyme that can elongate one or both of DNA 10 or nucleic acid probe 20. For example, nucleic acid probe 20 may be extended along the length of DNA 10 using a suitable polymerase enzyme, for instance, DNA pol. Other polymerases will be known to those of ordinary skill in the art. The extension of the probe may, for example, be used to ensure that the probe has been adequately bound to DNA 10, or to improve binding. In some cases, during elongation of the probe, a detection entity may be incorporated within the elongated nucleic acid, and/or attached to the elongated nucleic acid, as is illustrated in FIGS. 6A and 6B with detection entity 22.

Next, the DNA-probe hybrid is exposed to a methylation-sensitive restriction endonuclease that is able to cleave the DNA-probe hybrid only if methylation sites 16 and/or 26 are not methylated. Thus, in FIG. 6A, since both methylation sites 16 and 26 are methylated, the methylation-sensitive restriction endonuclease is not able to cleave the DNA-probe hybrid, and the hybrid thus remains unaltered. However, in FIG. 6B, since both methylation sites 16 and 26 are not methylated, the methylation-sensitive restriction endonuclease is able to bind to and cleave the DNA-probe hybrid, as is indicated by break 30.

Nucleic acid probe 20 can then be tested to determine whether the probe was cleaved or not. As shown in FIGS. 6A and 6B, tag sequence 28 on nucleic acid probe 20 is substantially complementary to a nucleic acid immobilized with respect to the service of array 40 at location 42. The DNA-probe hybrid may be denatured or melted to separate nucleic acid probe 20 from nucleic acid 10 and nucleic acid probe 20 and then exposed to the surface of array 40. Tag sequence 28 can become immobilized with respect to array 40 at location 42 by hybridizing the sequence to a substantially complementary nucleic acid immobilized at that location. Thus, in FIG. 6A, the entire nucleic acid probe 20, including detection entity 22, is localized to location 42. However, in FIG. 6D, only a fragment of nucleic acid probe 20, which does not contain detection entity 22, is immobilized with respect to location 42.

The presence or absence of detection entity 22 on array 40 with respect to location 42 can then be determined using any suitable technique, as previously noted. By determining the presence and/or concentration of detection entity 22 with respect to location 42, information can thereby be obtained as to whether methylation site 16 in DNA 10 was initially methylated or not.

As used herein, the term “determining” generally refers to the analysis of a species, for example, quantitatively or qualitatively, and/or the detection of the presence or absence of the species. “Determining” may also refer to the analysis of an interaction between two or more species, for example, quantitatively or qualitatively, and/or by detecting the presence or absence of the interaction. In addition, the terms “determining,” “measuring,” “evaluating,” “assessing,” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The target nucleic acid to be probed (e.g., DNA 10 in FIG. 1) may be any nucleic acid which includes, or is suspected to include, a methylation site. The nucleic acid may be, for example, DNA or RNA, and the nucleic acid may arise from any suitable source, for example, genomic DNA (which may be whole or fragmented, e.g., enzymatic ally and/or mechanically), mitochondrial DNA, cDNA, synthetic DNA, or the like. The target nucleic acid may have any suitable length. For example, the nucleic acid may have a length of at least about 10 nucleotides, at least about 25 nucleotides, at least about 40 nucleotides, at least about 50 nucleotides, at least about 75 nucleotides, at least about 100 nucleotides, at least about 300 nucleotides, at least about 1,000 nucleotides, at least about 10,000 nucleotides, at least about 100,000 nucleotides, etc. In some cases, for example, with genomic DNA, the nucleic acid may optionally first be cleaved, for instance, using chemicals or restriction endonucleases known to those of ordinary skill in the art, prior to determining methylation of the methylation site.

A “methylation site,” as used herein, is given its ordinary definition as used in the art, i.e., a base within a nucleic acid in which a hydrogen atom of the base can be enzymatically replaced by a methyl (—CH₃) group. The most common methylation site is the cytosine base of a “CpG” sequence within DNA, i.e., a cytosine followed by a guanine within the DNA strand (the “p” in the abbreviation “CpG” stands for the intervening phosphate between the two bases). Typically, the hydrogen in the “5” position of the cytosine is replaced by a methyl, forming 5-methylcytosine. CpG sequences have been linked to gene regulation, as well as changes or errors in gene expression, for example, in epigenetics or in cancer cells. In a nucleic acid duplex (two antiparallel strands associated at substantially complementary regions), if only one strand is methylated at a methylation site, the duplex is “hemi-methylated.” If both strands are methylated at the methylation site, the duplex is “fully methylated.” An example of a method of assessing CpG methylation is disclosed in U.S. Patent Application Publication No. 2005/0233340, published Oct. 20, 2005, entitled “Methods and Compositions for Assessing CpG Methylation,” by Barrett, et al., incorporated herein by reference.

CpG sequences within genomic DNA are often not randomly distributed, but are instead typically found in high concentrations in certain portions of the DNA, known as “CpG islands.” Some of the CpG islands have been linked to promoter sites. The CpG islands within DNA are generally rich in cytosine and guanine, some of which are located next to each other to form CpG pairs which are susceptible to methylation, as described above. However, in a CpG island, the cytosine and guanine residues do not necessarily have to occur at the same frequency or always be in a “CpG” repeat sequence. Those of ordinary skill in the art will be able to identify CpG islands within DNA. For instance, the CpG island may include at least about 50 nucleotides, and in some cases, the CpG island may include at least about 100 nucleotides or at least about 200 nucleotides. Within the CpG island, the frequency of appearance of cytosine and guanine may be significantly greater than chance (i.e., significantly greater than 25% for each, or 50% for both), and the frequency of each may be the same or different. For instance, within the CpG island, the combined frequency of cytosine and guanine may be at least about 60%, at least about 65%, at least about 70%, or at least about 75%, and cytosine and guanine may appear in the same or different percentages. As a non-limiting example, a CpG island may be identified as a region having between about 200 nucleotides and about 800 nucleotides, with a combined frequency of appearance of both cytosine and guanine greater than about 60% or about 65%.

As noted above, the subject oligonucleotides base pair with “CpG islands,” where a CpG island is defined as any discrete region of a genome that contains a CpG that is, or is predicted to be, a target for a cellular methyltransferase. CpG islands may be high-density CpG islands, such as those defined by Gardiner-Garden and Frommer, J. Mol. Biol., 1987;196:261-82, i.e., any stretch of DNA that is at least 200 bp in length that has a C+G content of at least 50% and an observed CpG/expected CpG ratio of greater than or equal to 0.60. CpG islands may also be low-density CpG islands, containing CpG dinucleotides that occur at a lower density in a given region. The methylation status of these low density CpG islands varies under different physiologic and pathologic conditions, including ageing and cancer, Toyota and Issa, Seminars in Cancer Biology, 1999;9:349-357. In general, CpG islands are generally found proximal to (i.e., within 1 kb, 3 kb, or about 5 kb of) the transcriptional start sites of eukaryotic genes. It has been estimated that there are approximately 45,000 CpG islands in the human genome and 37,000 CpG islands in the mouse genome (Antequera et al., Proc. Natl. Acad. Sci., 1993;90:11995-9.

A detailed discussion of CpG islands, methods for their identification, and many examples of CpG islands in human chromosomes is found in a variety of publications, including: Larsen et al., Genomics, 1992; 13:1095-1107; Takai et al., Proc. Natl. Acad. Sci., 2002;99:3740-3745; Antequera et al., Proc. Natl. Acad. Sci., 1993;90:11995-9; and Ioshikhes et al., Nat. Genet. 2000;26:61-3. Accordingly, CpG islands are well known in the art and need not be described herein in any more detail.

The CpG islands, due to the-presence of greater than normal C−G bonding, may have a melting temperature (“Tₘ”) that is substantially higher than the Tₘ of normal DNA (i.e., DNA in which adenine, cytosine, guanine, and thymine each appear with about equal frequency). The melting temperature may be defined as the temperature at which the nucleic acid duplex is 50% in single-standard form and 50% in double-standard form. Thus, for instance, the Tₘ of the DNA in a CpG island may be greater than about 60° C., greater than about 70° C., greater than about 75° C., greater than about 80° C., greater than about 85° C., greater than about 90° C., or greater than about 95° C., and in some cases, the DNA may not be readily analyzable using conventional techniques such as PCR, which often requires a melting temperature of between about 60° C. and about 75° C. Many prior art techniques for determining methylation of a nucleic acid thus cannot be effectively used to determine the methylation of nucleic acids containing CpG islands.

The nucleic acid to be probed may also include a “restriction site,” i.e. a site within the nucleic acid which is recognized by a restriction endonuclease, for example, a methylation-sensitive restriction endonuclease. Those of ordinary skill in the art will be familiar with restriction endonucleases, and restriction sites that are recognized by the restriction endonucleases. The restriction site may be located within the nucleic acid in a position such that the ability of a methylation-sensitive restriction endonuclease to cleave the nucleic acid may be altered by the presence or absence of a methyl group in a methylation site that is within or proximate to the recognition site, i.e., such that the presence of the methyl group in a methylation site alters the ability of the methylation-sensitive restriction endonuclease to cleave the nucleic acid even if the methylation site is not within the recognition site. Thus, in some cases, the restriction site may include the methylation site, for example, as depicted schematically in FIG. 1. However, in other cases, the restriction site may not necessarily include the methylation site, but may be in a position relatively close to the methylation site, as discussed in more detail below. The restriction site may have any appropriate size, as is known to those of ordinary skill in the art. For example, the restriction site may have a length of 4 base pairs, 6 base pairs, 8 base pairs, etc.

As mentioned, the target nucleic acid (e.g., DNA) is exposed to a nucleic acid probe (i.e., a probe able to bind a nucleic acid such as DNA) to determine the methylation state of a methylation site within the nucleic acid, i.e., whether the methylation site of the nucleic acid has been methylated or not. The nucleic acid probe may include a nucleic acid (e.g., DNA or RNA), which comprises naturally-occurring nucleotide bases. The probe may also include a hybridization region that recognizes at least a portion of the target nucleic acid to be probed, i.e., a region or sequence of the probe is substantially complementary to the nucleic acid. The nucleic acid probe may also include a tag sequence, and optionally, a detection entity, as discussed in more detail below. The hybridization region, methylation site, tag sequence, and detection entity (if present) may occur in any suitable order within the nucleic acid probe. In some cases, the nucleic acid may also comprise one, two, three, or more non-naturally-occurring nucleotide bases, which may, for instance, facilitate binding of detection entities, or be used to control the Tₘ of the probe.

As used herein, “substantially complementary,” in reference to two nucleic acids, means that the two nucleic acids each contain hybridization regions that are of sufficiently complementary as to be able to interact with each other in a specific, determinable fashion, i.e., when the two nucleic acids are brought together in an antiparallel orientation, the same nucleotides of each nucleic acid will become hybridized to each other at one or more specific locations (although both nucleic acids do not necessarily need to become completely hybridized to each other). The hybridization regions may be of a length that allows specific recognition. For example, the hybridization regions may be a length of at least about 10 nucleotides, at least about 15 nucleotides, at least about 20 nucleotides, at least about 25 nucleotides, at least about 30 nucleotides, at least about 40 nucleotides, at least about 50 nucleotides, or the like. In some cases, two hybridization regions that are substantially complementary to each other may be at least about 75% complementary, and in some cases, are at least about 80%, at least about 85%, at least about 90%, at least about 95%, at least about 96%, at least about 97%, at least about 98%, at least about 99%, at least about 99.5%, or 100% complementary to each other, e.g., via Watson-Click pairing (where every adenine within the hybridization region binds to thymine and vice versa, and every cytosine binds to guanosine and vice versa), and/or via analogous base-pairing with non-naturally occurring nucleotide bases. In some cases, the two nucleic acids that are sufficiently complementary in their hybridization regions may have a maximum of 40 mismatches in their hybridization regions (e.g., where one base of one nucleic acid does not have a complementary partner on the other nucleic acid, for example, due to additions, deletions, substitutions, bulges, etc.), and in other cases, the two hybridization regions may have a maximum of 30 mismatches, 20 mismatches, 10 mismatches, or 7 mismatches. In still other cases, the two hybridization regions may have a maximum of 6, 5, 4, 3, 2, 1, or 0 mismatches.

The hybridization region of the nucleic acid probe may be at least substantially complementary to the target nucleic acid in a portion of the nucleic acid that includes a methylation site suspected of being methylated, and/or a restriction site. As discussed above, the methylation site and the restriction site may be, but need not be, overlapping. In some cases, the hybridization region of the nucleic acid probe may also be substantially complementary to other portions of the target nucleic acid that are not part of the methylation site or the restriction site.

Additionally, the nucleic acid probe may include a detection entity, and/or a site for attachment of a detection entity. One non-limitating example of a detection entity is a fluorescent moiety. As used herein, a “detection entity” is an entity that is capable of indicating its existence in a particular sample or at a particular location. Detection entities of the invention can be those that are identifiable by the unaided human eye, those that may be invisible in isolation but may be detectable by the unaided human eye if in sufficient quantity, entities that absorb or emit electromagnetic radiation at a level or within a wavelength range such that they can be readily detected visibly (unaided or with a microscope including a fluorescence microscope or an electron microscope, or the like), spectroscopically, or the like. Non-limiting examples include fluorescent moieties (including phosphorescent moieties), fluorescent nucleotides, radioactive moieties, electron-dense moieties, dyes, chemiluminescent entities, electrochemiluminescent entities, enzyme-linked signaling moieties, etc. In some cases, the detection entity itself is not directly determined, but instead interacts with a second entity (a “signaling entity”) in order to effect determination; for example, coupling of the signaling entity to the detection entity may result in a determinable signal. The detection entity may be covalently attached to the nucleic acid probe as a separate entity (e.g., a fluorescent molecule), or the detection entity may be integrated within the nucleic acid, for example, covalently or as an intercalation entity, as a detectable sequence of nucleotides within the nucleic acid probe, etc. More than one detection entity may be used, and the detection entities may be distinguishable, i.e., the detection entities can be independently detected and measured, even when the detection entities are mixed. In other words, the amounts of detection entity present (e.g., the amount of fluorescence) for each of the detection entities can be separately determined, even when the labels are co-located (e.g., in the same tube or in the same duplex molecule or in the same feature of an array). Suitable distinguishable fluorescent label pairs include, but are not limited to, Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY VI 005 (Molecular Probes, Eugene, Oreg.), POPO-3 and TOTO-3 (Molecular Probes, Eugene, Oreg.), fluorescein and Texas red (Dupont, Bostan Mass.) and POPRO3 and TOPRO3 (Molecular Probes, Eugene, Oreg.). Further suitable detection entities are described in Kricka et al., Ann. Clin. Biochem., 2002;39:114-29, incorporated herein by reference.

In certain embodiments, the detection entity of the nucleic acid probe is not within the hybridization region, but may be positioned “upstream” or “downstream” of the hybridization region. However, in some cases, the detection entity is positioned relatively close to the restriction site, for example, such that there are less than 50 nucleotide, less than 40 nucleotides separating the restriction site from the methylation site, or in some cases, less than 30 nucleotides, less than 20 nucleotides, less than 15 nucleotides, less than 10 nucleotides, or less than 5 nucleotides separating the detection entity and the restriction site. In some cases, the restriction site and the methylation site may be adjacent or even overlapping.

The nucleic acid probe may also include a “tag” sequence, which may be used to identify the nucleic acid probe, for example, to distinguish the nucleic acid probe from other, similar nucleic acid probes. The tag sequence does not necessarily encode a protein or a peptide, and may be arbitrarily chosen in some cases. In one set of embodiments, the tag sequence is used to attach a nucleic acid probe to the surface of a substrate, for example, the surface of an array or the surface of a particle. In other embodiments, the tag sequence may be used to direct the nucleic acid probe to other reactions, etc. The tag sequence may be of any suitable length. For example, the tag sequence may have a length of about 50 nucleotides or less, about 40 nucleotides or less, about 30 nucleotides or less, about 20 nucleotides or less, about 10 nucleotides or less, or about 5 nucleotides or less. In some cases, the tag sequence may be positioned relatively close to the restriction site. For instance, the tag sequence and the restriction site may be adjacent or even overlapping, or separated by several intervening nucleotides, for instance, such that there are less than 50 nucleotides separating the restriction site from the methylation site, or in some cases, less than 40 nucleotides, less than 30 nucleotides, less than 20 nucleotides, less than 15 nucleotides, less than 10 nucleotides, or less than 5 nucleotides separating the tag sequence from the methylation site.

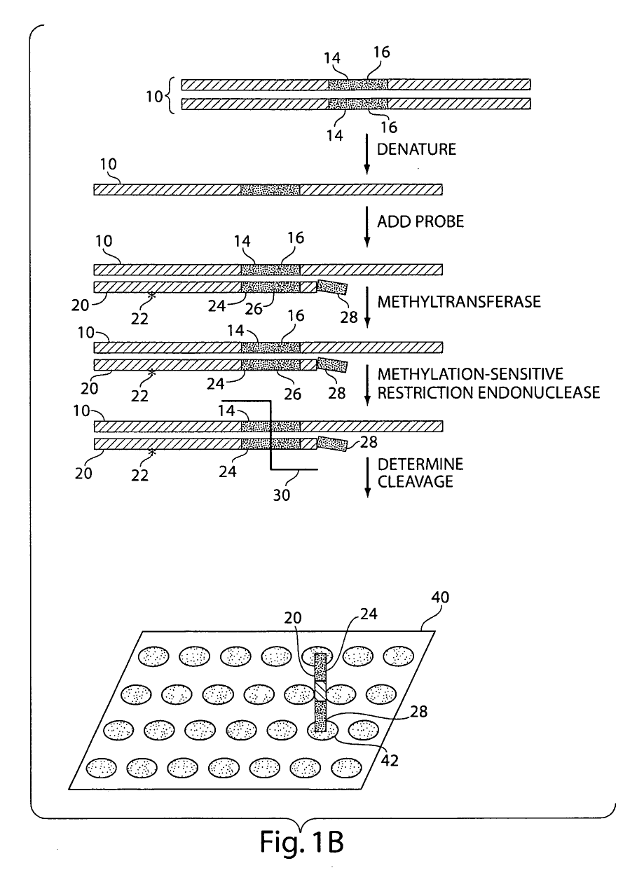

Thus, a non-limiting example of a nucleic acid probe of the invention is a probe having a tag sequence of about 40 nucleotides and a hybridization region having about 40 nucleotides to about 50 nucleotides, where the hybridization region is able to hybridize a target nucleic acid to be probed, and where the target nucleic acid includes a methylation site and a restriction site. The nucleic acid probe may include, within the hybridization region, sequences at least substantially complementary to the methylation site and/or the restriction site. Specific, non-limiting examples of nucleic acid probes are shown in FIGS. 2A and 2B, respectively. In each of these figures, a nucleic acid probe 50 is shown, comprising a restriction site (underlined) 54, a detection entity attachment site 52, and a tag sequence 58. In the interests of clarity, only the hybridization regions of the nucleic acid probes are shown in FIGS. 2A and 2B (SEQ ID NO: 1 and SEQ ID NO: 2, respectively); the tag sequences are not specifically shown in these examples, and are merely indicated as “TAG-a” and “TAG-b,” respectively. It should be noted that in this example, the hybridization region includes both restriction site 54 and site 52 for attachment of a detection entity.

The nucleic acid probe may be produced using any suitable method, for example, using de novo DNA synthesis techniques known to those of ordinary skill in the art, such as solid-phase DNA synthesis techniques, or U.S. patent application Ser. No. 11/234,701, filed Sep. 23, 2005, entitled “Methods for In Situ Generation of Nucleic Acid Molecules,” incorporated herein by reference. The probes may have a total length, for example, of at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, or at least 100 nucleotides.

The probe is then hybridized or annealed to the target nucleic acid to be probed to form a nucleic acid-nucleic acid probe hybrid. As described above, the nucleic acid probe may have a hybridization region that is substantially complementary to the target nucleic acid to be probed, and such a nucleic acid probe is then able to hybridize the target nucleic acid at least that portion, thereby forming the nucleic acid-nucleic acid probe hybrid. Hybridization can be performed under any suitable conditions. Suitable conditions for hybridizing nucleic acid sequences, at least a portion of which are substantially complimentary, are known to those of ordinary skill in the art. For example, suitable denaturing agents, or salt and/or buffer solutions in which to perform the hybridization reaction may be readily identified without undue effort. In some cases, such agents, salts, etc., may also be chosen to lower or otherwise alter the melting point (Tₘ) of the target nucleic acid. A non-limiting example of a suitable denaturing agent is formamide.

Typically, the hybridization is performed under conditions in which the target nucleic acid to be probed is single-stranded. Where double-stranded nucleic acids are used, e.g., in the case of double-stranded DNA, the double-stranded nucleic acid may be melted or denatured prior to, or simultaneously with, hybridization of the probe and the target nucleic acid.

As a non-limiting example, a mixture of a nucleic acid probe and a target nucleic acid may be heated to a temperature (of the mixture) that is at least sufficient to induce hybridization between the probe and the target nucleic acid, and preferably below temperatures which can cause the target nucleic acid to degrade. In some cases, the hybridization temperature is determined relative to the Tₘ of the target nucleic acid. For example, the mixture may be heated to a temperature greater than the Tₘ of the target nucleic acid, then cooled to facilitate hybridization. In some cases, temperatures lower than the Tₘ may be sufficient to cause hybridization. For example, the mixture may be heated to a temperature greater than about (Tₘ-25° C.), greater than about (Tₘ-20° C.), greater than about (Tₘ-15° C.), greater than about (Tₘ-10° C.), or greater than about (Tₘ-5° C.). In other cases, however, temperatures higher than the Tₘ of the target nucleic acid may be required. Thus, for example, the temperature of the mixture may be heated to a temperature of about 60° C., about 65° C., about 70° C., about 75° C., about 80° C., about 85° C., about 90° C., or about 95° C., then subsequently allowed to cool, for example, to 37° C., or to room temperature (about 25° C.).

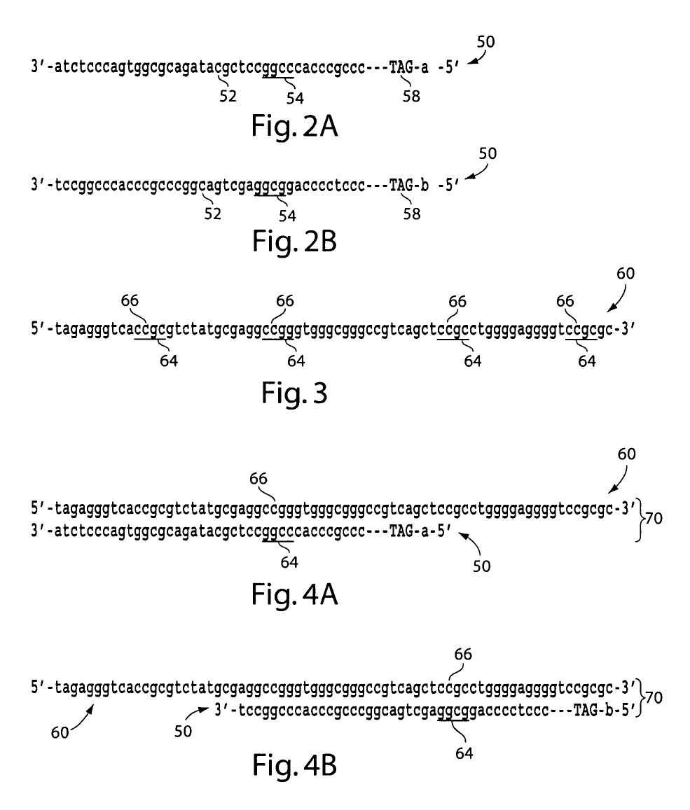

As a specific non-limiting example, if the portion of a genomic DNA sequence shown in FIG. 3 (SEQ ID NO: 3) is to be investigated, where the genomic sequence is suspected of containing one or more methylation sites, at least some of which are suspected of actually being methylated, probes such as those shown in FIGS. 2A and 2B may be used to investigate some of these methylation sites, as follows. In FIG. 3, DNA 60 contains a plurality of restriction sites 64, each of which sites contains a cytosine 66 that can be methylated. In DNA 60, the underlined sequence CCGC is the restriction site for the restriction endonuclease AciI, and the underlined sequence CCGG is the restriction site for the restriction endonuclease HpalI.

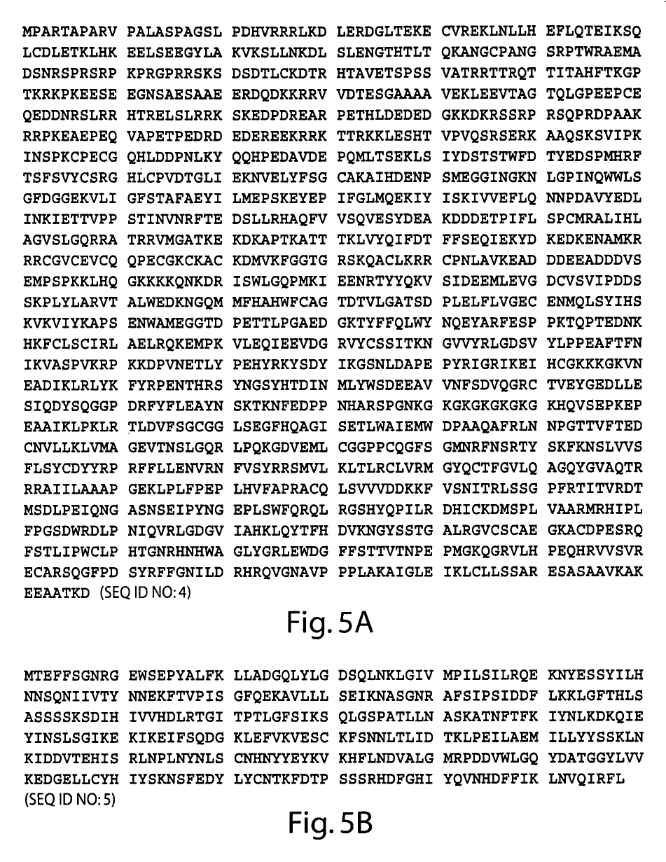

Nucleic acid probe 50, shown in FIG. 2A, can hybridize to DNA 60, as is illustrated in FIG. 4A, forming nucleic acid-nucleic acid probe hybrid 70. A portion of nucleic acid probe 50 is substantially complementary to DNA 60 and is shown adjacent to DNA 60, illustrating the complementarity of the two nucleic acid strands, while other portions of nucleic acid probe 50 (e.g., TAG-a) are not substantially complementary to DNA 60 and are not able to hybridize DNA 60. Similarly, in FIG. 4B, nucleic acid probe 50, as shown in FIG. 2B, can hybridize to DNA 60 in FIG. 3, forming nucleic acid-nucleic acid probe hybrid 70. In these figures, certain restriction sites are underlined. In FIG. 4A, the underlined restriction site 64 (GGCC) is recognized by the restriction endonuclease AciI, while in FIG. 4B, the underlined restriction site 64 (GGCG) is recognized by the restriction endonuclease HpaII.

In FIGS. 4A and 4B, a portion of each of the two example nucleic acid probes is substantially complimentary to a portion of DNA 60. However, it should be noted that the two nucleic acid probes do not hybridize to the same portion of DNA 60. Thus, as shown here, more than one methylation site of a nucleic acid can be examined, serially and/or simultaneously, depending on the nucleic acid probes selected to perform the analysis. In this example, the two nucleic acid probes are cleaved at different locations by different restriction endonucleases (AciI and HpaII, respectively), although in other embodiments, the same restriction endonuclease may be used to cleave two or more nucleic acid probes, more than one restriction endonuclease may be used to cleave a nucleic acid probe, etc.

Optionally, the nucleic acid-nucleic acid probe hybrid may be exposed to a methyltransferase, i.e., an enzyme able to catalyze the transfer of a methyl group located on one strand of a nucleic acid duplex to a complimentary strand. Thus, a hemi-methylated DNA-probe hybrid, i.e., a hybrid in which one of the DNA strands is methylated then becomes correspondingly methylated on the other strand, in approximately the same location, for example, CpG type that is methylated on one strand will become correspondingly methylated on the other strand of the DNA duplex, as the CpG site is self-complimentary. Non-limiting examples of C5-methylcytosine methyltransferases include DNMT1, DNMT2, DNMT3A, or DNMT3B. A source for methyl groups is also usually added, for example, S-adenosylmethionine (which release a methyl group to the methyltransferase to form S-adenosylhomocysteine).

Thus, if a methylation site on a target nucleic acid to be probed is methylated, then exposure of the nucleic acid-nucleic acid probe hybrid to the methyltransferase may “transfer” (i.e., copy) the methyl group from the nucleic acid to the complementary strand, i.e., to the nucleic acid probe, for example, as shown in FIG. 1A, i.e., converting a hemi-methylated hybrid into a fully methylated hybrid. Conversely, if the methylation site on the target nucleic acid to be probed is not methylated, then exposure of the nucleic acid-nucleic acid probe hybrid to the methyltransferase will not result in any alterations to the nucleic acid probe, and the nucleic acid probe will remain unmethylated at that location, for instance, as is shown in FIG. 1B.

The methyltransferase, as well as any methyl group sources, may be obtained from any suitable source. For example, the methyltransferase may be human methyltransferase, mouse methyltransferase, rat methyltransferase, or the like. Many methyltransferases and methyl group sources are commercially available, for example, from New England BioLabs, Ipswich, Mass.

In some embodiments, the nucleic acid-nucleic acid probe hybrid may be exposed to a polymerase, and such an exposure may be performed before or after exposure of the nucleic acid-nucleic acid probe hybrid to a methyltransferase (if performed), as described above. Exposure of the nucleic acid-nucleic acid probe hybrid may be used, for instance, to ensure that the nucleic acid probe is sufficiently bound to the nucleic acid. Non-limiting examples of polymerases include DNA pol I, DNA pol II, DNA pol III, DNA pol IV, DNA pol V, or DNA pol alpha, DNA pol beta, DNA pol gamma, DNA pol delta, DNA pol epsilon, or DNA pol zeta. Additional examples of polymerases include, but are not limited to, Taq, Pwo, Pfu, Vent, Deep Vent, Tfl, HotTub, Tth, etc, which are to known to those of ordinary skill in the art and are readily available.

The nucleic acid-nucleic acid probe hybrid can then be exposed to a restriction endonuclease, such as a methylation-sensitive restriction endonuclease, that is able to bind to at least a portion of the nucleic acid-nucleic acid probe hybrid at a restriction site, or a site on the nucleic acid which is recognized by the restriction endonuclease. In some cases, the restriction endonuclease is able to cleave the nucleic acid-nucleic acid probe hybrid. Thus, one or both of the target nucleic acid and the nucleic acid probe may be cleaved, resulting, in certain cases, in two (or more) portions, some or all of which may remain in a hybridized state. For instance, as a non-limiting example, in FIG. 1B, a hybrid comprising DNA 10 and nucleic acid probe 20 is cleaved into two separate portions by a restriction endonuclease, as indicated by break 30.

In some embodiments, the restriction endonuclease is sensitive to the physical state of the nucleic acid-nucleic acid probe hybrid, and in some cases, the restriction endonuclease is unable to cleave the hybrid if the hybrid is in a certain state. For instance, if a methylation-sensitive restriction endonuclease is used, the methylation-sensitive restriction endonuclease may be able to cleave the nucleic acid-nucleic acid probe hybrid if a methylation site on either or both the target nucleic acid and the nucleic acid probe is not methylated, but is unable to, or is generally inhibited from (i.e., at a much reduced rate), cleaving the nucleic acid-nucleic acid probe hybrid if a methylation site is methylated. For instance, the restriction endonuclease may be able to cleave the nucleic acid-nucleic acid probe hybrid even if the hybrid is methylated (fully or hemi-), but at a reduced rate, relative to the rate that the nucleic acid-nucleic acid probe hybrid is cleaved when the methylation site is not methylated. In some cases, the methylation-sensitive restriction endonuclease is unable to cleave the nucleic acid-nucleic acid probe hybrid if the hybrid is at least hemi-methylated (i.e., only one strand of the hybrid is methylated at a methylation site); in other cases, the methylation-sensitive restriction endonuclease is unable to cleave the nucleic acid-nucleic acid probe hybrid only if the hybrid is fully methylated (i.e., both strands of the hybrid are methylated at a methylation site).

If a methylation site is present, the methylation site and the restriction site may be positioned within the nucleic acid such that, if the methylation site is methylated, the restriction endonuclease is unable to bind to the restriction site, or is able to bind the restriction site, but is unable to cleave the nucleic acid-nucleic acid probe hybrid. For example, due to conformational effects, the ability of the restriction endonuclease to recognize the restriction site may be altered by the presence of the methyl group. Thus, the restriction site, in some embodiments, may include a methylation site, but in other embodiments, the restriction site and the methylation site may be separated. For example, the methylation site and the restriction site may be adjacent, or separated by several intervening nucleotides, for instance, such that there are less than 50 nucleotide, less than 40 nucleotides, less than 30 nucleotides separating the restriction site from the methylation site, or in some cases, less than 20 nucleotides, less than 15 nucleotides, less than 10 nucleotides, or less than 5 nucleotides separating the restriction site from the methylation site.

Non-limiting examples of methylation-sensitive restriction endonucleases include HpaII and AciI. Other non-limiting examples of potentially suitable methylation-sensitive restriction endonucleases include AarI, AatI, AatII, AccI, AccII, AccIII, Acc65I, AccB7I, AciI, AclI, AcuI, AdeI, AfaI, AfeI, AfII, AfIII, AfIIII, AgeI, AhaII, AhdI, AjnI, AleI, AloI, AluI, M.AluI, AlwI, Nt.AlwI, Alw21I, Alw26I, Alw44I, AlwNI, AmaI, AorI, Aor51HI, AosII, ApaI, ApaLI, ApeI, ApoI, ApyI, AquI, AscI, AseI, AsiSI, Asp700I, Asp718I, AspCNI, AspMI, AspMDI, AsuII, AtuSI, AvaI, AvaII, AviII, BaeI, BalI, BamFI, BamHI, M.BamHI, BamKI, BanI, BanII, BazI, BbeI, BbiII, BbrPI, BbsI, BbuI, BbvI, BbvCI, Bca77I, BccI, Bce243I, BceAI, BcgI, BciVI, BclI, BcnI, BepI, BfiI, Bfi57I, Bfi89I, BfrI, BfrBI, BfuI, BfuAI, BfuCI, BglI, BglII, BinI, BloHI, BlpI, BmaDI, Bme216I, Bme1390I, Bme1580I, BmeTI, BmeT110I, BmgBI, BmgT120I, BmrI, BmtI, BnaI, BoxI, BpiI, BplI, BpmI, BpuI, Bpu10I, Bpu1102I, BpuEI, BsaI, Bsa29I, BsaAI, BsaBI, BsaHI, BsaJI, BsaWI, BsaXI, BscI, BscFI, Bse634I, BseAI, BseCI, BseDI, BseGI, BseLI, BseMI, BseMII, BseRI, BseSI, BseXI, BseYI, BsgI, Bsh1236I, Bsh1285I, Bsh1365I, BshFI, BshGI, BshNI, BshTI, BsiBI, BsiEI, BsiHKAI, BsiLI, BsiMI, BsiQI, BsiSI, BsiWI, BsiXI, BslI, BsmI, BsmAI, BsmBI, BsmFI, BsoBI, BsoFI, Bsp49I, Bsp51I, Bsp52I, Bsp54I, Bsp56I, Bsp57I, Bsp58I, Bsp59I, Bsp60I, Bsp61I, Bsp64I, Bsp65I, Bsp66I, Bsp67I, Bsp68I, Bsp72I, Bsp91I, Bsp105I, Bsp106I, Bsp119I, Bsp120I, Bsp122I, Bsp143I, Bsp143II, Bsp1286I, Bsp2095I, BspAI, BspCNI, BspDI, Nt.BspD6I, BspEI, BspFI, BspHI, BspJ64I, BspKT6I, BspLI, BspLU11III, BspMI, BspMII, BspPI, BspRI, BspST5I, BspT104I, BspT107I, BspXI, BspXII, BspZEI, BsrI, BsrBI, BsrBRI, BsrDI, BsrFI, BsrPII, BssAI, BssHII, BssKI, BssSI, BstI, Bst1107I, BstAPI, BstBI, BstEII, BstEIII, BstENII, BstF5I, BstGI, BstKTI, BstNI, M.BstNI, Nt.BstNBI, BstOI, BstPI, BstSCI, BstUI, Bst2UI, BstVI, BstXI, BstYI, BstZ17I, Bsu15I, Bsu36I, BsuBI, BsuEII, BsuFI, BsuMI, BsuRI, BsuTUI, BtcI, BtgI, BtgZI, BtrI, BtsI, CacI, Cac8I, Cail, CauII, CbiI, CboI, CbrI, CceI, CcrI, CcyI, CfoI, CfrI, Cfr6I, Cfr9I, Cfr10I, Cfr13I, Cfr42I, CfrBI, CfuI, ClaI, CpeI, CpfI, CpfAI, CpoI, CspI, Csp5I, Csp6I, Csp45I, CspAI, Csp68KII, CthII, CtyI, CviAI, CviAII, CviBI, M.CviBIII, CviJI, Nt.CviPII, CviQI, Nt.CviQXI, CviRI, CviRII, CviSIII, DdeI, DpnI, DpnII, DraI, DraII, DraIII, DrdI, DsaV, EaeI, EagI, Eam1104I, Eam1105I, EarI, EcaI, EciI, Ecl136II, EclXI, Ecl18kI, Eco24I, Eco31I, Eco32I, Eco47I, Eco47III, Eco52I, Eco57I, Eco72I, Eco88I, Eco91I, Eco105I, Eco147I, Eco1831I, EcoAI, EcoBI, EcoDI, EcoHI, EcoHK31I, EcoKI, M.EcoKDam, EcoNI, EcoO65I, EcoO109I, EcoPI, EcoP15I, EcoRI, M.EcoRI, EcoRII, M.EcoRII, EcoRV, EcoR124I, EcoR124II, EcoT22I, EheI, EsaBC3I, EsaBC4I, EsaLHCI, Esp3I, Esp1396I, FatI, Faul, FbaI, FnuDII, FnuEI, Fnu4HI, FokI, M.FokI, FseI, FspI, FspAI, Fsp4HI, Gstl588II, GsuI, HaeII, HaeIII, M.HaeIII, HaeIV, HapII, HgaI, HgiAI, HgiCI, HgiCII, HgiDI, HgiEI, HgiHI, HhaI, HhaII, M.HhaII, Hin1I, Hin6I, HinP1I, HincII, HindII, HindIII, HinfI, HpaI, HpaII, M.HpaII, HphI, M1.HphI, Hpy8I, Hpy99I, Hpy99II, Hpy188I, Hpy188III, HpyAIII, HpyAIV, HpyCH4III, HpyCH4IV, HpyCH4V, HsoI, ItaI, KasI, KpnI, Kpn2I, KspI, Ksp22I, KspAI, KzQ9I, LlaAI, LlaKR2I, MabI, MaeII, MamI, MbiI, MboI, MboII, M1.MboII, Mel3JI, Mel5JI, Mel7JI, Mel4OI, Mel5OI, Mel2TI, Mel5TI, MfeI, MfII, MlsI, MluI, Mlu9273I, Mlu9273II, MlyI, MmeI, MmeII, Mmu5I, MmuP2I, MnlI, MpsI, MroI, MscI, MseI, MslI, MspI, M.MspI, MspA1I, MspBI, MspR9I, MssI, MstII, MthTI, MthZI, MunI, MvaI, Mva1269I, MvnI, MwoI, NaeI, NanII, NarI, NciI, NciAI, NcoI, NcuI, NdeI, NdeII, NgoBV, NgoBVIII, NgoCI, NgoCII, NgoFVII, NgoMIV, NgoPII, NgoSII, NgoWI, NheI, NlaIII, NlaIV, NlaX, NmeSI, NmuCI, NmuDI, NmuEI, NotI, NruI, NsbI, NsiI, NspI, NspV, NspBII, NspHI, PacI, PaeI, PaeR7I, PagI, PauI, PbrTI, PciI, PdiI, PdmI, Pei9403I, PfaI, Pfl23II, PflFI, PflMI, PfoI, PhoI, PleI, Ple19I, PmaCI, PmeI, PmlI, PpiI, PpuMI, Pru2I, PshAI, PsiI, Psp5II, Psp39I, Psp1406I, PspGI, PspOMI, PspPI, PstI, PsuI, PsyI, PvuI, PvuII, Ral8I, RaIF40I, RflFI, RflFII, Rrh4273I, RsaI, RshI, RspXI, RsrI, RsrII, SacI, SacII, SalI, SalDI, SapI, Sau961, Sau3239I, Sau3AI, SauLPI, SauMI, SbfI, Sbo13I, ScaI, Scg2I, SchI, ScrFI, SdaI, SduI, SenPI, SexAI, SfaNI, SfiI, SfoI, SfuI, SgfI, SgrAI, SgrBI, SinI, SlaI, SmaI, SmlI, SnaBI, SnoI, SolI, SpeI, SphI, SplI, SpoI, SrfI, Sru30DI, SscL1I, Sse9I, Sse8387I, SseBI, SsoI, SsoII, SspI, SspRFI, SstI, SstII, Sth302I, Sth368I, StsI, StuI, StyD4I, StyLTI, StyLTIII, StySJI, StySPI, StySQI, SuaI, SwaI, TaaI, TaiI, TaqI, M.TaqI, TaqII, TaqXI, TfiI, TflI, ThaI, TliI, TrsKTI, TrsSI, TrsTI, TseI, Tsp45I, Tsp509I, TspMI, TspRI, Tth111I, TthHB8I, Van91I, VpaK11BI, VspI, M.VspI, XapI, XbaI, XceI, XcmI, XcyI, XhoI, XhoII, XmaI, XmaIII, XmiI, XmnI, XorII, XspI, ZanI, or ZraI. Many of these methylation-sensitive restriction endonucleases are commercially available. For example, HpaII and Acil are available from New England Biolabs (Ipswich, Mass.). In some cases, more than one methylation-sensitive restriction endonuclease may be used, and the restriction endonucleases may recognize the same and/or different restriction sites on either one or both of the target nucleic acid and the nucleic acid probe.

The cleavage state of the nucleic acid probe is then determined, i.e., whether the nucleic acid probe is intact relative to the nucleic acid probe that the original target nucleic acid to be probed was exposed to, or whether the probe has been cleaved into one or more fragments. The cleavage state of the nucleic acid probe can be determined, in some cases, while the nucleic acid probe is still hybridized to the target nucleic acid. In other cases, however, the nucleic acid probe may be separated from the target nucleic acid, for example, by denaturing or melting as previously described, before determining the cleavage state of the nucleic acid probe.

In one set of embodiments, the cleavage state of the nucleic acid probe is determined by determining a detection entity attached to the nucleic acid probe, e.g., whether the detection entity is still attached to the entire nucleic acid probe, or is attached only to a portion of the probe. In one set of embodiments, as previously discussed, the nucleic acid probe, before exposure to the nucleic acid to be probed, includes a detection entity; however, in other embodiments, the nucleic acid probe does not contain a detection entity upon exposure to the nucleic acid to be probed, and the detection entity is added after hybridization, for example, before, during, or after exposure to the restriction endonuclease. In general, a target composition may be labeled using methods that are well known in the art (e.g., primer extension, random-priming, nick translation, etc.; see, e.g., Ausubel et al., Short Protocols in Molecular Biology, 3rd ed., Wiley & Sons 1995; or Sambrook et al., Molecular Cloning: A Laboratory Manual, Third Edition, 2001 Cold Spring Harbor, N.Y.), and, accordingly, such methods do not need to be described here in great detail. In particular embodiments, the target composition can be labeled with a fluorescence label. In some embodiments, the methods of labeling a nucleic acid probe with a detection entity generally follow the methods that are well known in the art and described in, e.g., Pinkel et al., Nat. Genet., 1998;20:207-211; Hodgson et al., Nat. Genet. 2001;29:459-464); and Wilhelm et al., Cancer Res., 2002;62: 957-960.

In one embodiment, the nucleic acid probe is attached to a surface at a first end (e.g., using a tag sequence, such as previously described), and the presence or absence of a detection entity on the nucleic acid probe (i.e., if the detection entity has not been subsequently cleaved off) on the surface is then determined. In such an embodiment, the nucleic acid probe can be attached to the surface before, during, or after hybridization of the target nucleic acid to the nucleic acid probe. In some cases, e.g., as shown in FIGS. 1A-1B, the nucleic acid probe can be attached to the surface after the nucleic acid-nucleic acid probe hybrid has been exposed to a restriction endonuclease. In addition, as further discussed below, the surface may include more than one type of nucleic acid probe, which may recognize the same or different target nucleic acids, and/or may recognize the same or different portions of a target nucleic acid. As mentioned, however, in other embodiments of the invention, a surface is not necessarily required in order to determine the cleavage state of the nucleic acid probe.

Thus, as an example, if a nucleic acid probe contains a tag sequence and a detection entity, separated by a restriction site, cleavage of the restriction site may cause separation of the tag sequence and the detection entity and any suitable method may be used to determine whether cleavage has occurred. As a specific example, in FIG. 1B, detection entity 22 on nucleic acid probe 20 is separated from tag sequence 28 by restriction site 26, such that cleavage of the nucleic acid probe separates the portion of the nucleic acid probe containing tag sequence 28 from the portion of the nucleic acid probe containing detection entity 22.

Of course, other methods may be used to determine the cleavage state of the nucleic acid probe, e.g., without necessarily requiring that the nucleic acid probe be attached to a surface. For instance, a nucleic acid probe may contain a first detection entity and a second detection entity, and the association of the first and second detection entities may be determined in some fashion, for example, in embodiments where the first and second detection entities are able to interact in a fashion that can be determined. Such a nucleic acid probe, in some cases, may not necessarily contain a tag sequence, i.e., the nucleic acid probe may contain a hybridization region, the methylation site, a first detection entity, and a second detection entity, and these may occur in any suitable order within the nucleic acid probe.

The tag sequence (which may or may not be associated with a surface) may be directly or indirectly determined, and the association of the detection entity with respect to the tag sequence may be used to determine the cleavage state of the nucleic acid probe. As an example, the molecular weight and/or the sequence of the nucleic acid probe may be determined, for example, using standard techniques such as gel electrophoresis, ultracentrifugation, mass spectroscopy, or the like, and the cleavage state of the nucleic acid probe may be correspondingly determined. Such a nucleic acid probe thus may not contain a detection entity and/or a tag sequence.

In another set of embodiments, the probe may be labeled using an enzyme able to participate in an enzymatic reaction. For example, the detection entity may be an enzyme such as Taq or klenow, for example, to produce a fluorescent signal or an otherwise determinable signal. Thus, if the detection entity is present on the probe, then reaction of the enzyme may produce a signal; however, if the detection entity is not present (e.g., due to cleavage), then no determinable signal may be produced.

In certain aspects, one or more types of nucleic acid probes may be attached to a surface. The nucleic acid probes may recognize the same or different nucleic acids, or may recognize the same or different portions of a nucleic acid sequence. The surface may be any suitable surface in which a nucleic acid probe may be attached, for example, the surface of a substrate, the surface of a particle, etc. In one set of embodiments, the surface is the surface of an array. Those of ordinary skill in the art will be familiar with the operation and use of arrays, i.e., a surface having a collection of microscopic elements or “spots,” which may be used to immobilize one or more compounds such as nucleic acid probes, as described in detail below. The elements on the substrate may be arranged in any suitable arrangement, for example, in a rectangular grid. The elements may be chosen to possess, or are chemically derivatized to possess, at least one reactive chemical group that can be used for further attachment chemistry, e.g., for attachment of a nucleic acid and/or a nucleic acid probe to the surface of the array. Such attachment may be covalent or non-covalent. There may also be optional molecular linkers interposed between the substrate and the reactive chemical groups used for molecular attachment.

The nucleic acids and/or the nucleic acid probes may be immobilized relative to a surface, e.g., the surface of an array, using any suitable technique known to those of ordinary skill in the art, for example, via chemical attachment (e.g., via covalent bonding), via one or more linkers bonded to the surface of the array (to which a nucleic acid or nucleic acid probe can bind), via non-covalent interactions, etc. In one set of embodiments, a linker may comprise one or more nucleic acids, and in some cases, at least a portion of the linker may comprise a hybridization region that is substantially complementary to a portion of a nucleic acid or a nucleic acid probe. For example, in one embodiment, the linker comprises a hybridization region that is substantially complementary to a tag sequence on a nucleic acid probe. If more than one nucleic acid probe is used, e.g., in an assay, the linkers may each comprise the same or different hybridization regions, for example, such that a first nucleic acid probe is able to bind a first linker (but not a second linker) and a second nucleic acid probe is able to bind the second linker (but not the first linker). Such discrimination may be achieved, for example, by using different tag sequences within the various nucleic acid probes, and such different tag sequences may be arbitrarily chosen in some instances. If an array is used, the linkers may be in the same or different elements or spots within the array.

The nucleic acids and/or the nucleic acid probes may be attached to surface before an assay is performed using the nucleic acids and/or nucleic acid probes, during, or afterwards. For example, in one embodiment, one or more nucleic acid probes may be immobilized relative to a surface, for instance, to one or more elements of an array, and subsequently exposed to one or more target nucleic acids to be probed. Hybridization of the nucleic acids and the nucleic acid probes may result in a number of nucleic acid-nucleic acid probe hybrids immobilized relative to the surface. The hybrids are then exposed to one or more restriction endonucleases, and the cleavage state of the hybrids can then be determined, e.g., whether the hybrids, or portions of the hybrids, remains immobilized relative to the surface.

In another embodiment, a nucleic acid probe may be used to determine methylation of a target nucleic acid by hybridizing the target nucleic acid probe to the nucleic acid, exposing the nucleic acid-nucleic acid probe hybrid to a restriction endonuclease, and then immobilizing the nucleic acid probe relative to a surface, for example, using a tag sequence on the nucleic acid probe. The cleavage state of the immobilized nucleic acid probe can then be determined.

In yet another embodiment, a nucleic acid is first immobilized relative to a surface, such as the surface of an array. For instance, a target nucleic acid may be immobilized relative to a surface, then exposed to a nucleic acid probe. Hybridization of the target nucleic acid and the nucleic acid probes may result in a number of nucleic acid-nucleic acid probe hybrids immobilized relative to the surface. The hybrids are then exposed to a restriction endonuclease, and the cleavage state of the probes is then determined. In still another embodiment, hybridization of a target nucleic acid and a nucleic acid probe may be performed prior to immobilizing the target nucleic acid relative to a surface.

In one set of embodiments of the invention, more than one nucleic acid probe may be used to determine the methylation state of one or more methylation sites on a target nucleic acid to be probed. For example, one or more nucleic acid probes may be attached to a surface, such as the surface of an array, for instance, relative to different elements where each tag sequence of each nucleic acid probe is associated with a different element of the array. By determining the cleavage states of the nucleic acid probes associated with the elements of the array, methylation of the nucleic acid can be determined. For example, a first element on a array may be used to indicate the methylation state of a first methylation site, while a second element on the array may be used to indicate the methylation state of a second methylation site of the nucleic acid to be probed, or the same methylation site but under different physical conditions.

It should be noted that the systems and methods of the invention are as described herein not limited only to determining methylation of a nucleic acid, but can be used to determine other physical conditions of certain target sites of target nucleic acids. Accordingly, it is to be understood that the above-described systems and methods, in connection with determining methylation of a target nucleic acid, are by way of example only. In other aspects, a target nucleic acid to be probed may have a target site that can be in one of a plurality of states, some or all of which may be naturally occurring in some embodiments of the invention. For example, the target site may be a site suspected of being a phosphorylation site, a SNP (single nucleotide polymorphism) site, or the like. One or more nucleic acid probes may be prepared that are able to hybridize the target nucleic acid proximate the target site. The nucleic acid-nucleic acid probe hybrid may then be exposed to a restriction endonuclease that is not able to cleave (or is generally inhibited from cleaving) the nucleic acid if the target site of the nucleic acid is in a first state, but is able to cleave the nucleic acid if the target site is in a second state different from the first state. After exposure of the nucleic acid-nucleic probe hybrid to the restriction endonuclease, the cleavage state of the nucleic acid probe may be determined, and used to determine the state of the target site, i.e., if the nucleic acid probe has been cleaved, the target site may be in a first state, and if the nucleic acid probe is not cleaved, then the target site may be at a second state, etc.

Another aspect of the invention is generally directed to a kit. A “kit,” as used herein, typically defines a package including one or more of the compositions of the invention, and/or other compositions associated with the invention, for example, a nucleic acid probe, as previously described. For example, the kit may include, in one set of embodiments, one or more nucleic acid probes, as described herein, optionally in combination within an array, such as is described in more detail below. The kit may be directed to determining the methylation of one or more selected nucleic acids molecules, for example, of genomic DNA, mitochondrial DNA, etc. More than one type of nucleic acid probe may be included within the kit, in some cases, and the probes may be labeled or unlabeled with detection entities. In one embodiment, the nucleic acid probes may correspond to specific or predetermined locations on the array, for example, the array may contain sequences that are complimentary to sequences within the nucleic acid probe, for example, as is illustrated in FIG. I with nucleic acid probe 20 and location 42 of array 40. The kits may also include one or more control analyte mixtures, e.g., two or more control compositions for use in testing the kit.

Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, modifying, assembling, storing, packaging, preparing, mixing, diluting, and/or preserving the compositions components for a particular use.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, assembly, storage, packaging, and/or preparation of the compositions and/or other compositions associated with the kit. In some cases, the instructions may also include instructions, for example, for a particular use. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

Unless defined otherwise, all technical and scientific terms used herein have the same meaning as commonly understood by one of ordinary skill in the art to which this invention belongs. Still, certain terms are defined below for the sake of clarity and ease of reference.

The term “sample,” as used herein, relates to a material or mixture of materials, typically, although not necessarily, in fluid form, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “mixture,” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of surface-bound polynucleotides, as is commonly known in the art and described herein, is not a mixture of surface-bound polynucleotides because the species of surface-bound polynucleotides are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “biomolecule” means any organic or biochemical molecule, group or species of interest that may be formed in an array on a substrate surface. Non-limiting examples of biomolecules include peptides, proteins, amino acids, and nucleic acids.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source. A “biomonomer” refers to a single unit, which can be linked with the same or other biomonomers to form a biopolymer (e.g., a single amino acid or nucleotide with two linking groups, one or both of which may have removable protecting groups). A biomonomer fluid or biopolymer fluid reference a liquid containing either a biomonomer or biopolymer, respectively (typically in solution).

The term “peptide,” as used herein, refers to any compound produced by amide formation between a carboxyl group of one amino acid and an amino group of another group. The term “oligopeptide,” as used herein, refers to peptides with fewer than about 10 to 20 residues, i.e., amino acid monomeric units. As used herein, the term “polypeptide” refers to peptides with more than 10 to 20 residues. The term “protein,” as used herein, refers to polypeptides of specific sequence of more than about 50 residues.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art, synthesis of nucleic acids of this type may utilize, in some cases, an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include, but are non limited to, deoxyribonucleotides (DNA), ribonucleotides (RNA), or other polynucleotides which are C-glycosides of a purine or pyrimidine base. The oligomer may be defined by, for example, about 2-500 monomers, about 10-500 monomers, or about 50-250 monomers.

The terms “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively). The terms “ribonucleic acid” and “RNA,” as used herein, refer to a polymer comprising ribonucleotides. The terms “deoxyribonucleic acid” and “DNA,” as used herein, mean a polymer comprising deoxyribonucleotides. The term “oligonucleotide” as used herein denotes single stranded nucleotide multimers of from about 10 to 200 nucleotides and up to about 500 nucleotides in length. For instance, the oligonucleotide may be greater than about 60 nucleotides, greater than about 100 nucleotides or greater than about 150 nucleotides.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides. Examples of naturally occurring bases within the nucleotide include adenosine or “A,” thymidine or “T,” guanosine or “G,” cytidine or “C,” or uridine or “U.” Examples of non-naturally occurring bases include, but are not limited to, 2-aminoadenosine, 2-thiothymidine, inosine, pyrrolopyrimidine, 3-methyladenosine, C5-bromouridine, C5-fluorouridine, C5-iodouridine, C5-propynyluridine, C5-propynylcytidine, C5-methylcytidine, 7-deazaadenosine, 7-deazaguanosine, 8-oxoadenosine, 8-oxoguanosine, O6-methylguanosine, 2-thiocytidine, 2-aminopurine, 2-amino-6-chloropurine, 2,6-diaminopurine, or hypoxanthine.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. Generally, as used herein, the terms “oligonucleotide” and “polynucleotide” are used interchangeably. Further, generally, the term “nucleic acid” or “nucleic acid molecule” also encompasses oligonucleotides and polynucleotides.

The phrase “labeled population of nucleic acids” refers to mixture of nucleic acids that are detectably labeled, e.g., fluorescently labeled, such that the presence of the nucleic acids can be detected by assessing the presence of the label. A labeled population of nucleic acids can be “made from” a “CpG island composition” or a “sample composition.” The composition may be employed as template for making the population of nucleic acids in some cases.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements, and amplification of any subchromosomal region or DNA sequence. In certain embodiments, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

If a surface-bound nucleic acid or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array elements, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

The terms “hybridize” or “hybridization,” as is known to those of ordinary skill in the art, refer to the binding or duplexing of a nucleic acid molecule to a particular nucleotide sequence under suitable conditions, e.g., under stringent conditions. “Hybridizing” and “binding,” with respect to nucleic acids, are used interchangeably. The above hybridization step may also include agitation, where the agitation may be accomplished using any convenient protocol, e.g., shaking, rotating, spinning, and the like.

The term “stringent conditions” (or “stringent hybridization conditions”) as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent conditions are the summation or combination (totality) of both hybridization and wash conditions.

Stringent conditions (e.g., as in array, Southern or Northern hybridizations) may be sequence dependent, and are often different under different experimental parameters. Stringent conditions that can be used to hybridize nucleic acids include, for instance, hybridization in a buffer comprising 50% formamide, 5×SSC (salt, sodium citrate), and 1% SDS at 42° C., or hybridization in a buffer comprising 5×SSC and 1% SDS at 65° C., both with a wash of 0.2×SSC and 0.1% SDS at 65° C. Other examples of stringent conditions include a hybridization in a buffer of 40% formamide, 1 M NaCl, and 1% SDS at 37° C., and a wash in 1×SSC at 45° C. In another example, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional examples of stringent conditions include hybridization at 60° C. or higher and 3×SSC (450 mM sodium chloride/45 mM sodium citrate) or incubation at 42° C. in a solution containing 30% formamide, 1 M NaCl, 0.5% sodium lauryl sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to another nucleic acid (for example, when a nucleic acid has hybridized to a nucleic acid probe). Wash conditions used to identify nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (e.g., for 14-base oligos), 48° C. (e.g., for 17-base oligos), 55° C. (e.g., for 20-base oligos), or 60° C. (e.g., for 23-base oligos). See Sambrook, Ausubel, or Tijssen (cited elsewhere herein) for detailed descriptions of equilvalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences and reduce the complexity of the sample prior to hybridization. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Additional hybridization methods are described in references describing CGH techniques (Kallioniemi etal., Science, 1992;258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, e.g., Gall et al., Meth. Enzymol., 1981;21:470-480 and Angerer et al., In Genetic Engineering. Principles and Methods, Setlow and Hollaender, Eds. Vol 7, pgs 43-65 (Plenum Press, New York 1985). See also U.S. Pat. Nos. 6,335,167, 6,197,501, 5,830,645, and 5,665,549, the disclosures of which are herein incorporated by reference.

The phrases “nucleic acid molecule bound to a surface of a solid support,” “probe bound to a solid support,” “probe immobilized with respect to a surface,” “target bound to a solid support,” or “polynucleotide bound to a solid support” (and similar terms) generally refer to a nucleic acid molecule (e.g., an oligonucleotide or polynucleotide) or a mimetic thereof (e.g., comprising at least one PNA, UNA, and/or LNA monomer) that is immobilized on the surface of a solid substrate, where the substrate can have a variety of configurations, e.g., including, but not limited to, planar substrates, non-planar substrate, a sheet, bead, particle, slide, wafer, web, fiber, tube, capillary, microfluidic channel or reservoir, or other structure. The solid support may be porous or non-porous. In certain embodiments, collections of nucleic acid molecules are present on a surface of the same support, e.g., in the form of an array, which can include at least about two nucleic acid molecules. The two or more nucleic acid molecules may be identical or comprise a different nucleotide base composition.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any one or more of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5″ terminus). In some cases, the arrays are arrays of polypeptides, e.g., proteins or fragments thereof. The term “array” also encompasses the term “microarray.”

The substrate may be formed in essentially any shape. In one set of embodiments, the substrate has at least one surface which is substantially planar. However, in other embodiments, the substrate may also include indentations, protuberances, steps, ridges, terraces, or the like. The substrate may be formed from any suitable material, depending upon the application. For example, the substrate may be a silicon-based chip or a glass slide. Other suitable substrate materials for the arrays of the present invention include, but are not limited to, glasses, ceramics, plastics, metals, alloys, carbon, agarose, silica, quartz, cellulose, polyacrylamide, polyamide, polyimide, and gelatin, as well as other polymer supports or other solid-material supports. Polymers that may be used in the substrate include, but are not limited to, polystyrene, poly(tetra)fluoroethylene (PTFE), polyvinylidenedifluoride, polycarbonate, polymethylmethacrylate, polyvinylethylene, polyethyleneimine, polyoxymethylene (POM), polyvinylphenol, polylactides, polymethacrylimide (PMI), polyalkenesulfone (PAS), polypropylene, polyethylene, polyhydroxyethylmethacrylate (HEMA), polydimethylsiloxane, polyacrylamide, polyimide, various block co-polymers, etc.

Any given substrate may carry any number of oligonucleotides on a surface thereof. In some cases, one, two, three, four, or more arrays may be disposed on a surface of the substrate. Depending upon the use, any or all of the arrays may be the same or different from one another and each may contain multiple spots, or elements or features. A typical array may contain more than two, more than ten, more than one hundred, more than one thousand more ten thousand features, or even more than one hundred thousand features, in an area of less than 20 cm² or even less than 10 cm² As mentioned, however, in other embodiments of the invention, a surface is not necessarily required in order to determine the cleavage state of the nucleic acid probe. For example, features may have widths (that is, diameter, for a round spot) in the range from a 10 micrometers to 1.0 cm. In other embodiments each feature may have a width in the range of 1.0 micrometers to 1.0 mm, 5.0 micrometers to 500 micrometers, 10 micrometers to 200 micrometers, etc. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20%, 50%, 75%, 90%, 95%, 99%, or 100% of the total number of features). Interfeature areas may be present in some embodiments which do not carry any oligonucleotide (or other biopolymer or chemical moiety of a type of which the features are composed). Such interfeature areas may be present where the arrays are formed by processes involving drop deposition of reagents but may not be present when, for example, light directed synthesis fabrication processes are used. It will be appreciated though, that the interfeature areas, when present, could be of various sizes and configurations.

The substrate may have thereon a pattern of locations (or elements) (e.g., rows and columns) or may be unpatterned or comprise a random pattern. The elements may each independently be the same or different. For example, in certain cases, at least about 25% of the elements are substantially identical (e.g., comprise the same sequence composition and length). In certain other cases, at least 50% of the elements are substantially identical, or at least about 75% of the elements are substantially identical. In certain cases, some or all of the elements are completely or at least substantially identical. For instance, if nucleic acids are immobilized on the surface of a solid substrate, at least about 25%, at least about 50%, or at least about 75% of the oligonucleotides may have the same length, and in some cases, may be substantially identical.

An “array layout” or “array characteristics,” refers to one or more physical, chemical or biological characteristics of the array, such as positioning of some or all the features within the array and on a substrate, one or more dimensions of the spots or elements, or some indication of an identity or function (for example, chemical or biological) of a moiety at a given location, or how the array should be handled (for example, conditions under which the array is exposed to a sample, or array reading specifications or controls following sample exposure).

Each array may cover an area of less than 200 cm², or even less than 100 cm², less than 50 cm², 10 cm², 1 cm², 0.5 cm² or 1 cm² In certain embodiments, the substrate carrying the one or more arrays will be shaped as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. In some cases, the substrate will have a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. In some instances, with arrays that are read by detecting fluorescence, the substrate may be of a material that emits low fluorescence upon illumination with the excitation light. Additionally, in some cases the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident thereon, as may be measured across the entire integrated spectrum of such illuminating light or alternatively at 532 nm or 633 nm.

In certain embodiments, a nucleic acid sequence may be present as a composition of multiple copies of the nucleic acid molecule on the surface of the array, e.g., as a spot or element on the surface of the substrate. The spots may be present as a pattern, where the pattern may be in the form of organized rows and columns of spots, e.g., a grid of spots, across the substrate surface, a series of curvilinear rows across the substrate surface, e.g., a series of concentric circles or semi-circles of spots, or the like. The density of spots present on the array surface may vary, for example, at least about 10, at least about 100 spots/cm², at least about 1,000 spots/cm², or at least about 10,000 spots/cm². In other embodiments, however, the elements are not arranged in the form of distinct spots, but may be positioned on the surface such that there is substantially no space separating one element from another.

In some embodiments, the array may be referred to as addressable. An array is “addressable” when it has multiple regions of different moieties (e.g., different nucleic acids) such that a region (i.e., an element or “spot” of the array) at a particular predetermined location (i.e., an “address”) on the array may be used to detect a particular target or class of targets (although an element may incidentally detect non-targets of that element). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., nucleic acid molecules, to be evaluated by binding with the other). In the present application, the “population of labeled nucleic acids” or “sample composition” and the like will be referenced as a moiety in a mobile phase, to be detected by “surface-bound polynucleotides” which are bound to the substrate at the various regions. These phrases are synonymous with the arbitrary terms “target” and “probe,” or “probe” and “target,” respectively, as they are used in other publications.

A “scan region” refers to a contiguous (preferably, rectangular) area in which the array spots or elements of interest, as discussed above, are found. For example, the scan region may be that portion of the total area illuminated from which resulting fluorescence is detected and recorded. For the purposes of this invention, the scan region includes the entire area of the slide scanned in each pass of the lens, between the first element of interest, and the last element of interest, even if there exist intervening areas which lack elements of interest. An “array layout” refers to one or more characteristics of the features, such as element positioning on the substrate, one or more feature dimensions, and an indication of a moiety at a given location.

In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

The arrays can be fabricated using drop deposition from pulsejets of either oligonucleotide precursor units (such as monomers) in the case of in situ fabrication, or the previously obtained oligonucleotide. Such methods are described in detail in, for example, in U.S. Pat. Nos. 6,242,266, 6,232,072, 6,180,351, 6,171,797, or 6,323,043, or in U.S. patent application Ser. No. 09/302,898, filed Apr. 30, 1999, and the references cited therein. These references are each incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

In using an array made by the method of the present invention, the array will be exposed in certain embodiments to a sample (for example, a fluorescently labeled target nucleic acid molecule) and the array then read. Reading of the array may be accomplished, for instance, by illuminating the array and reading the location and intensity of resulting fluorescence at various locations of the array (e.g., at each spot or element) to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. Pat. Nos. 6,756,202 or 6,406,849, each incorporated herein by reference. Other suitable devices and methods are described in U.S. patent application Ser. No. 09/846,125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels), or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

Arrays may also be read by any other method or apparatus than the foregoing, with other reading methods, including other optical techniques (for example, detecting chemiluminescent or electroluminescent labels) or electrical techniques (where each feature is provided with an electrode to detect hybridization at that feature in a manner disclosed in, e.g., U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. All definitions, as defined and used herein, should be understood to control over dictionary definitions, definitions in documents incorporated by reference, and/or ordinary meanings of the defined terms.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. In this specification and the appended claims, the singular forms “a,” “an” and “the” include plural reference unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

“Optional” or “optionally,” as used herein, means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

It should also be understood that, unless clearly indicated to the contrary, in any methods claimed herein that include more than one step or act, the order of the steps or acts of the method is not necessarily limited to the order in which the steps or acts of the method are recited.

All publications mentioned herein are incorporated herein by reference for the purpose of describing and disclosing the invention components that are described in the publications that might be used in connection with the presently described invention.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of determining methylation of a nucleic acid molecule, comprising acts of:
providing a nucleic acid molecule suspected of being methylated at a methylation site;
hybridizing a nucleic acid probe to the nucleic acid molecule proximate the methylation site to produce a nucleic acid molecule-nucleic acid probe hybrid;
exposing the nucleic acid-nucleic acid probe hybrid to a methyltransferase;
exposing the nucleic acid molecule-nucleic acid probe hybrid to a methylation-sensitive restriction endonuclease; and
determining a cleavage state of the nucleic acid probe to determine methylation of the nucleic acid at the methylation site.

2. The method of claim 1, wherein the nucleic acid is DNA.

3. The method of claim 1, wherein the methylation-sensitive restriction endonuclease is an enzyme selected from the group consisting of HpaII and Acil.

4. The method of claim 1, wherein the methyltransferase methylates hemi-methylated double stranded nucleic acids.

5. The method of claim 4, wherein the methyltransferase is DnmtI.

6. The method of claim 1, wherein the nucleic acid probe is fluorescently labeled, and the act of determining the cleavage state of the nucleic acid probe comprises detecting the presence or absence of the fluorescent label of the nucleic acid probe.

7. The method of claim 1, further comprising, prior to the act of exposing the nucleic acid-nucleic acid probe hybrid to the methylation-sensitive restriction endonuclease, immobilizing a fluorescent entity with respect to the nucleic acid probe.

8. The method of claim 1, wherein the nucleic acid comprises a restriction site, recognized by the methylation-sensitive restriction endonuclease, that is within 50 base pairs of the methylation site.

9. The method of claim 1, wherein the methylation site is contained within a restriction site of the nucleic acid that is recognized by the methylation-sensitive restriction endonuclease.

10. The method of claim 1, wherein the nucleic acid probe hybridizes to at least a portion of a CpG island contained within the nucleic acid.

11. The method of claim IO, wherein the CpG island contained within the nucleic acid comprises the methylation site.

12. The method of claim 1, wherein the nucleic acid has a Tₘ of at least about 70° C.

13. The method of claim 1, wherein the nucleic acid arises from genomic DNA.

14. The method of claim 1, wherein the nucleic acid arises from fragmented genomic DNA.

15. The method of claim 1, wherein the nucleic acid arises from mitochondrial DNA.

16. The method of claim 1, wherein the nucleic acid probe is contacted to a nucleic acid array.

17. The method of claim 1, comprising exposing the nucleic acid molecule suspected of being methylated to a plurality of non-identical nucleic acid probes.

18. The method of claim 17, wherein at least two of the plurality of non-identical nucleic acid probes are each able to hybridize to different portions of the nucleic acid molecule.

19. The method of claim 1, wherein the nucleic acid probe comprises a detection entity.

20. The method of claim 1, the nucleic acid probe further comprising a tag sequence, wherein the act of determining a cleavage state of the nucleic acid probe comprises binding the tag sequence of the nucleic acid probe to an array.

21. The method of claim 1, wherein the nucleic acid probe further comprises a methylation site.

22. The method of claim 1, wherein the nucleic acid probe further comprises a restriction site.

23. The method of claim 22, wherein the restriction site further comprises a methylation site.

24. A method of determining methylation of a nucleic acid molecule, comprising acts of:
exposing a nucleic acid molecule to a surface having at least a first region comprising a first nucleic acid probe immobilized thereto and a second region comprising a second nucleic acid probe immobilized thereto, wherein the first nucleic acid probe is able to hybridize the nucleic acid molecule at a first region suspected of being methylated at a first methylation site, and the second nucleic acid probe is able to hybridize the nucleic acid molecule at a second region suspected of being methylated at a second methylation site different from the first methylation site;
exposing at least one of the first nucleic acid probe and the second nucleic acid probe to a restriction endonuclease; and
determining a cleavage state of the first nucleic acid probe and/or the second nucleic acid probe to determine, respectively, methylation of the nucleic acid at the first methylation site and/or the second methylation site.

25. A method of determining the state of a target site of nucleic acid, comprising acts of:
providing a nucleic acid molecule having a target site that can be in one of a plurality of naturally-occurring states, including a first state and a second state;
hybridizing a nucleic acid probe to the nucleic acid molecule proximate the target site;
exposing the nucleic acid-nucleic acid probe hybrid to a restriction endonuclease that does not bind the nucleic acid molecule if the target site is in a first state, but does bind the nucleic acid if the target site is in a second state; and
thereafter, determining a cleavage state of the nucleic acid probe to determine the state of the target site.

26. A kit for determining methylation of a nucleic acid molecule, the kit comprising:
a nucleic acid probe comprising a hybridization region, a restriction site comprising a methylation site, and a detection entity; and
a methylation-sensitive restriction endonuclease.


SEQUENCE LISTING (5 sequences)

SEQ ID NO: 1 - DNA, 39 bp, Artificial Sequence
  Synthetic Sequence
atctcccagtggcgcagatacgctccggcccacccgccc

SEQ ID NO: 2 - DNA, 39 bp, Artificial Sequence
  Synthetic Sequence
tccggcccacccgcccggcagtcgaggcggacccctccc

SEQ ID NO: 3 - DNA, 70 bp, Artificial Sequence
  Synthetic Sequence
tagagggtcaccgcgtctatgcgaggccgggtgggcgggccgtcagctccgcctggggaggggtccgcgc

SEQ ID NO: 4 - protein, 1627 aa, Mus musculus
MPARTAPARVPALASPAGSLPDHVRRRLKDLERDGLTEKECVREKLNLLHEFLQTEIKSQLCDLETKLHKEELSEEGYLAKVKSLLNKDLSLENGTHTLTQKANGCPANGSRPTWRAEMADSNRSPRSRPKPRGPRRSKSDSDTLCKDTRHTAVETSPSSVATRRTTRQTTITAHFTKGPTKRKPKEESEEGNSAESAAEERDQDKKRRVVDTESGAAAAVEKLEEVTAGTQLGPEEPCEQEDDNRSLRRHTRELSLRRKSKEDPDREARPETHLDEDEDGKKDKRSSRPRSQPRDPAAKRRPKEAEPEQVAPETPEDRDEDEREEKRRKTTRKKLESHTVPVQSRSERKAAQSKSVIPKINSPKCPECGQHLDDPNLKYQQHPEDAVDEPQMLTSEKLSIYDSTSTWFDTYEDSPMHRFTSFSVYCSRGHLCPVDTGLIEKNVELYFSGCAKAIHDENPSMEGGINGKNLGPINQWWLSGFDGGEKVLIGFSTAFAEYILMEPSKEYEPIFGLMQEKIYISKIVVEFLQNNPDAVYEDLINKIETTVPPSTINVNRFTEDSLLRHAQFVVSQVESYDEAKDDDETPIFLSPCMRALIHLAGVSLGQRRATRRVMGATKEKDKAPTKATTTKLVYQIFDTFFSEQIEKYDKEDKENAMKRRRCGVCEVCQQPECGKCKACKDMVKFGGTGRSKQACLKRRCPNLAVKEADDDEEADDDVSEMPSPKKLHQGKKKKQNKDRISWLGQPMKIEENRTYYQKVSIDEEMLEVGDCVSVIPDDSSKPLYLARVTALWEDKNGQMMFHAHWFCAGTDTVLGATSDPLELFLVGECENMQLSYIHSKVKVIYKAPSENWAMEGGTDPETTLPGAEDGKTYFFQLWYNQEYARFESPPKTQPTEDNKHKFCLSCIRLAELRQKEMPKVLEQIEEVDGRVYCSSITKNGVVYRLGDSVYLPPEAFTFNIKVASPVKRPKKDPVNETLYPEHYRKYSDYIKGSNLDAPEPYRIGRIKEIHCGKKKGKVNEADIKLRLYKFYRPENTHRSYNGSYHTDINMLYWSDEEAVVNFSDVQGRCTVEYGEDLLESIQDYSQGGPDRFYFLEAYNSKTKNFEDPPNHARSPGNKGKGKGKGKGKGKHQVSEPKEPEAAIKLPKLRTLDVFSGCGGLSEGFHQAGISETLWAIEMWDPAAQAFRLNNPGTTVFTEDCNVLLKLVMAGEVTNSLGQRLPQKGDVEMLCGGPPCQGFSGMNRFNSRTYSKFKNSLVVSFLSYCDYYRPRFFLLENVRNFVSYRRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEIQNGASNSEIPYNGEPLSWFQRQLRGSHYQPILRDHICKDMSPLVAARMRHIPLFPGSDWRDLPNIQVRLGDGVIAHKLQYTFHDVKNGYSSTGALRGVCSCAEGKACDPESRQFSTLIPWCLPHTGNRHNHWAGLYGRLEWDGFFSTTVTNPEPMGKQGRVLHPEQHRVVSVRECARSQGFPDSYRFFGNILDRHRQVGNAVPPPLAKAIGLEIKLCLLSSARESASAAVKAKEEAATKD

SEQ ID NO: 5 - protein, 358 aa, Haemophilus parainfluenzae
MTEFFSGNRGEWSEPYALFKLLADGQLYLGDSQLNKLGIVMPILSILRQEKNYESSYILHNNSQNIIVTYNNEKFTVPISGFQEKAVLLLSEIKNASGNRAFSIPSIDDFLKKLGFTHLSASSSSKSDIHIVVHDLRTGITPTLGFSIKSQLGSPATLLNASKATNFTFKIYNLKDKQIEYINSLSGIKEKIKEIFSQDGKLEFVKVESCKFSNNLTLIDTKLPEILAEMILLYYSSKLNKIDDVTEHISRLNPLNYNLSCNHNYYEYKVKHFLNDVALGMRPDDVWLGQYDATGGYLVVKEDGELLCYHIYSKNSFEDYLYCNTKFDTPSSSRHDFGHIYQVNHDFFIKLNVQIRFL